(19) United States
(12) Patent Application Publication
(10) Pub. No.: US 2023/0020802 A1
(43) Pub. Date: Jan. 19, 2023

(54) SYSTEMS, METHODS, AND DEVICES FOR PRESSURE ADJUSTMENT USING A SYRINGE HOLDER

(71) Applicant: PRONK TECHNOLOGIES, INC., Sun Valley, CA (US)

(72) Inventor: Karl A. Ruiter, Honolulu, HI (US)

(21) Appl. No.: 17/866,376

(22) Filed: Jul. 15, 2022

Related U.S. Application Data
(60) Provisional application No. 63/222,886, filed on Jul. 16, 2021.

(51) Int. Cl. A61M 5/315; A61M 5/31

(52) U.S. Cl. CPC A61M 5/31515; A61M 5/3129; A61M 2205/3331; A61M 2205/70

(57) ABSTRACT

Systems, devices, and methods for a syringe holder. A system embodiment may include: a base platform defining a syringe flange opening and a set of adjustment openings, a latch assembly movably attached to the base platform, the latch assembly comprising an adjustment screw threaded between the base platform and the latch assembly, and a guidepost oriented parallel to the adjustment screw, wherein the guidepost is threaded between the base platform and the latch assembly, a valve assembly attached to the base platform, and a hose attached to the base platform.

### CROSS-REFERENCE TO RELATED APPLICATION

This application claims the priority benefit of U.S. Provisional Patent Application Ser. No. 63/222,886, filed Jul. 16, 2021, all of which are hereby incorporated herein by reference in their entirety for all purposes.

### TECHNICAL FIELD

Embodiments relate generally to measuring pressure and more specifically to pressure testing, pressure calibration, and pressure adjustment.

### BACKGROUND

Many devices in the medical field and other technical fields measure a pressure or vacuum value. These devices require periodic testing and calibration, which requires the operator the physically produce precise pressure values. Syringes are frequently used for a variety of applications in the medical environment. While a bare syringe is capable of creating a pressure or vacuum, its limited precision and capacity leaves it unsuitable for many pressure testing applications.

### SUMMARY

A system embodiment may include: a base platform defining a syringe flange opening configured to receive a syringe barrel flange of a syringe; and a latch assembly configured to movably attach to the base platform via an adjustment screw threaded between the base platform and the latch assembly, where a distance between the base platform and the latch assembly may be changed based on a rotation of the adjustment screw relative to the latch assembly, the latch assembly comprising: a handle configured to rotate relative to the latch assembly; and a latch securing fastener configured to engage a rib of a syringe plunger by rotation of the handle; and where the rib of the syringe plunger may be configured to be secured to the latch assembly via rotation of the handle of the latch assembly; and where rotation of the adjustment screw may be configured to generate a precise pressure within the syringe by moving the syringe plunger relative to the syringe barrel flange, when the rib of the syringe plunger may be secured to the latch assembly.

In additional system embodiments, an increase in the distance between the base platform and the latch assembly may be configured to decrease a pressure value of the syringe. In additional system embodiments, a decrease in the distance between the base platform and the latch assembly may be configured to increase a pressure value of the syringe.

Additional system embodiments may further include: a guidepost further configured to secure the base platform to the latch assembly, where the guidepost runs through a guidepost opening of the base platform and a guidepost opening of the latch assembly. In additional system embodiments, the rib of the syringe plunger may be configured to be released via rotation of the handle of the latch assembly. In additional system embodiments, the handle may be configured to rotate relative to the latch assembly to move a latch securing fastener at least one of: toward the rib and away from the rib.

In additional system embodiments, the latch securing fastener comprises threads configured to mate with threads disposed in a portion of the latch assembly. In additional system embodiments, the adjustment screw comprises a threaded shaft that runs through at least a portion of the base platform and at least a portion of the latch assembly.

Additional system embodiments may further include: a hose configured to be attached to an end of the syringe; and a hose attachment member on the base platform, where the hose attachment member may be configured to receive an end of the hose. Additional system embodiments may further include: a valve assembly configured to be attached to an end of the syringe; and a valve assembly attachment member on the base platform, where the valve assembly attachment member may be configured to receive a portion of the valve assembly.

A method embodiment may include: securing a syringe barrel flange of a syringe to a base platform; securing a latch assembly to the base platform via an adjustment screw threaded between at least a portion of the base platform and at least a portion of the latch assembly; securing a rib of a syringe plunger of the disposable syringe to the latch assembly; and rotating the adjustment screw to adjust a distance between the syringe plunger and the syringe barrel flange to generate a precise pressure within the syringe.

In additional method embodiments, rotation of the adjustment screw adjusts a distance between the base platform and the latch assembly. In additional method embodiments, securing the rib of the syringe plunger of the disposable syringe to the latch assembly further comprises: rotating a handle relative to the latch assembly, where a latch securing fastener may be configured to engage the rib of the syringe plunger by rotation of the handle.

Additional method embodiments may further include: decreasing a pressure value of the syringe by increasing a distance between the base platform and the latch assembly. Additional method embodiments may further include: increasing a pressure value of the syringe by decreasing a distance between the base platform and the latch assembly.

Additional method embodiments may further include: releasing the rib of the syringe plunger of the disposable syringe to the latch assembly. Additional method embodiments may further include: detaching a valve assembly from a valve assembly attachment member on the base platform; and securing the valve assembly to an end of the syringe. Additional method embodiments may further include: detaching a hose from a hose attachment member on the base platform; and securing the hose to an end of the syringe.

Another method embodiment may include: securing a syringe barrel flange of a syringe to a base platform; securing a latch assembly to the base platform via an adjustment screw threaded between at least a portion of the base platform and at least a portion of the latch assembly; moving a syringe plunger relative to the syringe barrel flange to a distance between the syringe plunger and the syringe barrel flange to generate an approximate pressure within the syringe; securing a rib of a syringe plunger of the disposable syringe to the latch assembly; and rotating the adjustment screw to further adjust the distance between the syringe plunger and the syringe barrel flange to generate a precise pressure within the syringe. In additional method embodiments, rotation of the adjustment screw adjusts a distance between the base platform and the latch assembly.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts a perspective view of a pressure adjustment system, according to an embodiment;

FIG. 2 depicts a rear view of the pressure adjustment system, according to an embodiment;

FIG. 3 depicts a front view of the pressure adjustment system, according to an embodiment;

FIG. 4 depicts a left-side view of the pressure adjustment system, according to an embodiment;

FIG. 5 depicts a magnified view of a valve assembly of the pressure adjustment system, according to an embodiment; FIG. 6 is a high-level block diagram of the pressure adjustment system, according to an embodiment;

FIG. 7 is a flowchart for a method embodiment of using the pressure adjustment system, according to an embodiment; and

FIG. 8 is a flowchart for a method embodiment of setting up the pressure adjustment system, according to an embodiment.

### DETAILED DESCRIPTION

The present system, device, and method disclosed herein works in conjunction with a syringe to make an assembly that is broadly useful for pressure or vacuum testing. The present pressure adjustment system disclosed herein allows for a disposable syringe to be used as a pump or a vacuum to generate pressure for a pump. The syringe holder systems, devices, and methods disclosed herein allow a user to better hold the flanges of the syringe barrel in order to apply more pressure to the syringe plunger. In some embodiments, the present syringe holder system may use inserted screws to help secure the syringe barrel to a more stable object such as a workbench. The disclosed system may be made of a stiff plastic such as polycarbonate. Other materials are possible and contemplated.

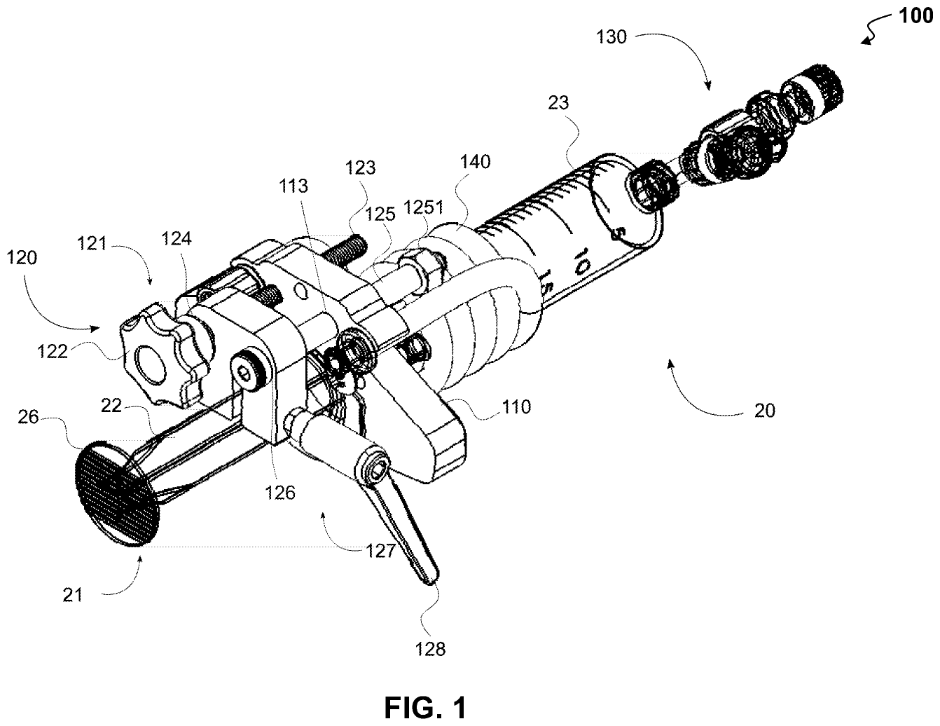

According to one embodiment depicted in FIG. 1, a pressure adjustment system 100 used to hold a syringe 20 may include a base platform 110, a latch assembly 120 movably connected to the base platform 110, a valve assembly 130 connected to an end of the syringe 20, and a hose 140 connected to the base platform 110. The latch assembly 120 may be configured to detachably attach to one or more of the set of ribs 22 distal a plunger flange 26 of the syringe plunger 21 by rotation of a handle 128 of the latch assembly 120. The ribs 22 may be structural supports, e.g., in the shape of fins, for the syringe plunger 21. The rotation of the handle 128 pushes a latch securing fastener 127 into contact with one or more of the set of ribs 22, thus securing the latch assembly 120 to the syringe plunger 21 by, for example, frictional fit. The latch securing fastener 127 may include threads that mate with corresponding threads in a portion of the latch assembly 120. Rotating the handle 128 in a first direction may cause the threads of the latch securing fastener 127 to engage with corresponding threads in a portion of the latch assembly 120 to move the latch securing fastener 127 towards the ribs 22. Rotating the handle 128 in a second direction may cause the threads of the latch securing fastener 127 to engage with corresponding threads in a portion of the latch assembly 120 to move the latch securing fastener 127 towards the ribs 22. The first direction may be clockwise and the second direction may be counterclockwise in one embodiment. The first direction may be counterclockwise and the second direction may be clockwise in another embodiment. While a handle 128 and latch securing fastener 127 are shown, other methods of securing the latch assembly 120 to the syringe plunger 21 are possible and contemplated. In some embodiments, a plate may be attached to one or more of the set of ribs 22. The plate may include indents where a screw or other fasteners may engage the indent to secure the plate to one or more of the set of ribs 22.

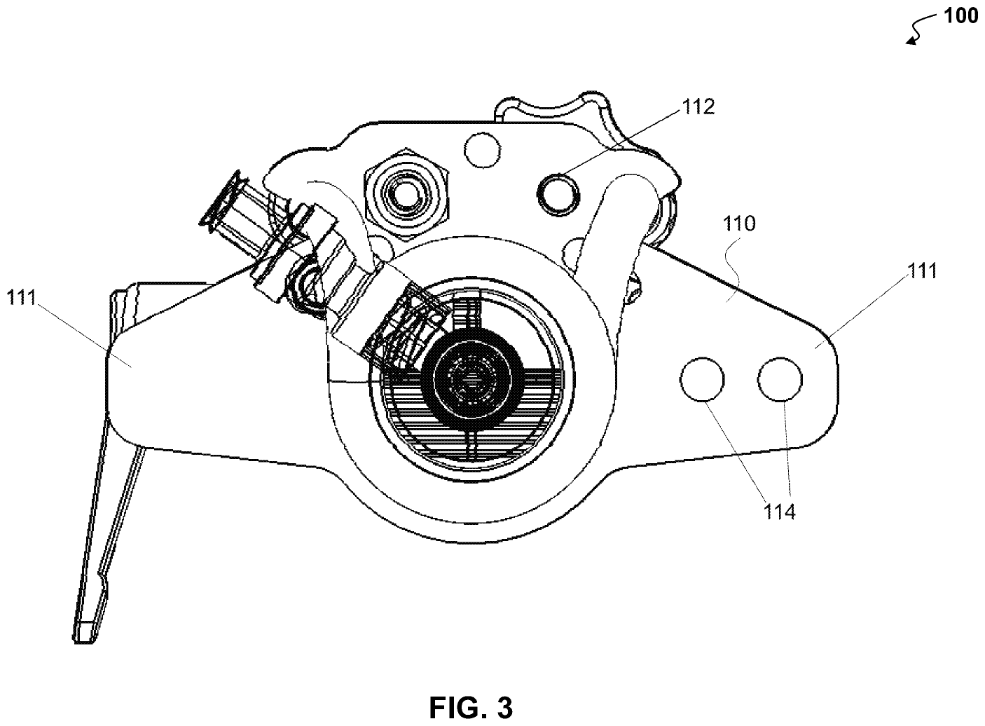

With reference to FIGS. 1 and 3, the latch assembly 120 may move relative to the base platform 110 by rotating an adjustment screw 121 using a knob 122. The distance between the base platform 110 and the latch assembly 120 may be increased or decreased based on the rotation of the adjustment screw 121 via knob 122. The knob 122 may be rotated to ensure that the syringe plunger 21 moves relative to the syringe 20 such that a desired precision pressure value or vacuum value is achieved within the syringe barrel 23.

The adjustment screw 121 may include a threaded shaft 123 that runs through at least a portion of the base platform 110 and at least a portion of the latch assembly 120. In some embodiments, the latch assembly 120 may have at least one shaft opening or aperture 124, where the shaft may be threaded, allowing the latch assembly 120 to slide relative to the base platform 110 with each rotation of the knob 122. In some embodiments, a corresponding shaft opening 112 in the base platform 110 may be threaded. In other embodiments, the shaft opening 124 in the latch assembly 120 may be threaded. For example, rotation of the knob 122 in a clockwise direction may cause the distance between the latch assembly 120 and the base platform 110 to decrease, which causes the attached syringe plunger 21 to push inward relative to the syringe 20 thereby increasing the pressure value or vacuum value. Likewise, rotation of the knob 122 in a counter-clockwise direction may cause the distance between the latch assembly 120 and the base platform 110 to increase, which causes the attached syringe plunger 21 to pull outward relative to the syringe 20 thereby decreasing the pressure value or vacuum value.

According to one embodiment, the present pressure adjustment system 100 provides a mechanism where the adjustment screw 121 enables a user to make fine, granular, and/or slight adjustments to ensure that the syringe plunger 21 moves a desired amount relative to the syringe 20 to make the pressure value or vacuum value precisely correct. In some embodiments, the base platform 110 may include a threaded opening 112 (see FIG. 3) for receiving the threaded shaft 123. In some embodiments the threaded shaft 123 may also further include one or more stoppers, such as nuts, placed on the threaded shaft 123 between the latch assembly 120 and the base platform 110 to assure that rotating the threaded shaft 123 in a counterclockwise direction will decrease the pressure value or generate a vacuum. In some embodiments, the one or more stoppers may include a slot and a clip combination or a shoulder. A double nut may be used as a stopper to allow for the use of a standard sized screw. In some embodiments, a clip may be used as one or more stoppers and a slot may be machined into the threaded shaft 123 to allow the clip to be inserted therein. In some embodiments, the threaded shaft 123 may include a ring and/or a shoulder machined onto the threaded shaft 123 to act as a stopper. Other stopper variations are possible and contemplated.

The latch assembly 120 may be partially secured to the base platform 110 by a guidepost 125 oriented to be parallel with the threaded shaft 123 as viewed along a local plane of the structure. The guidepost 125 and threaded shaft 123 may be oriented in a plane substantially perpendicular to the latch assembly 120 and the base platform 110. The guidepost 125 may run through both a guidepost opening 113 of the base platform 110 and a guidepost opening 126 of the latch assembly 120. The guidepost 125 may have one or more nuts 1251 to prevent either the base platform 110 or the latch assembly 120 from sliding off the guidepost 125. In some embodiments, the guidepost 125 may have an aperture drilled crosswise through an end of the guidepost 125 for insertion of a pin to prevent either the base platform 110 or the latch assembly 120 from sliding off the guidepost 125. In other embodiments, the guidepost 125 may include a flare on an end to prevent either the base platform 110 or the latch assembly 120 from sliding off the guidepost 125.

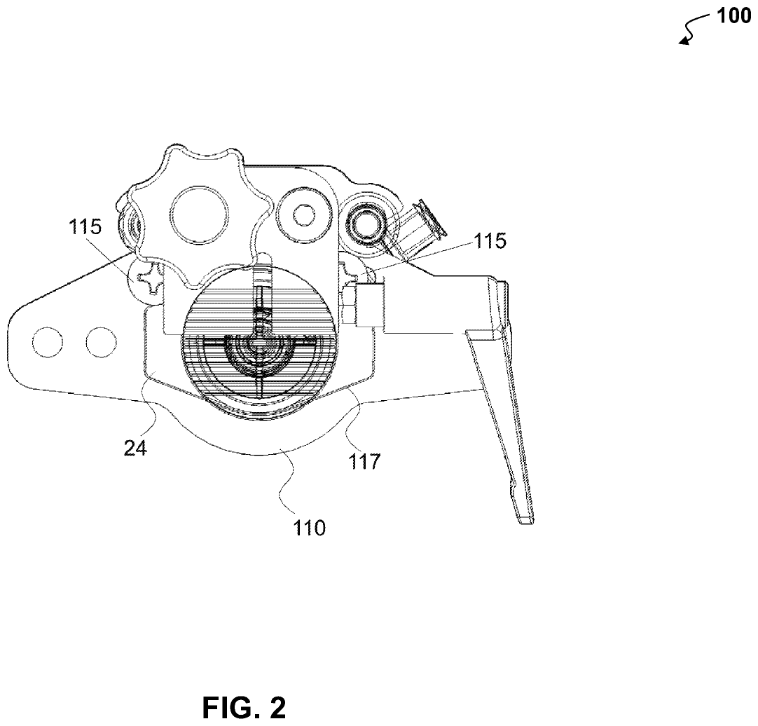

FIG. 2 depicts a rear view of the syringe holder system 100 and FIG. 3 depicts a front view of the syringe holder system 100. Referring to FIGS. 2-3, the base platform 110 defines a syringe flange opening 117 and a set of clamp attachment openings 114 on the wings 111 of the base platform 110. The clamp attachment openings 114 may be configured to attach the base platform 110 to a clamp. The clamp may be configured to hold the disclosed assembly to an external object, such as a workbench. The wings 111 may, for example, be engaged by a clamp on a workbench to secure the present syringe holder system 100 to the workbench. The syringe barrel flange 24 is secured within the syringe flange opening 117 by threading a set of base securing fasteners 115 (see FIG. 2) into the set of clamp attachment openings 114. The base securing fasteners 115 are configured to attach the flange of the syringe barrel (ref. no. 23, FIG. 1) to the base platform 110. The set of syringe barrel flange securing openings 117 may be positioned along the wings 111 to correspond to the different shapes and sizes of the syringe barrel flange 24.

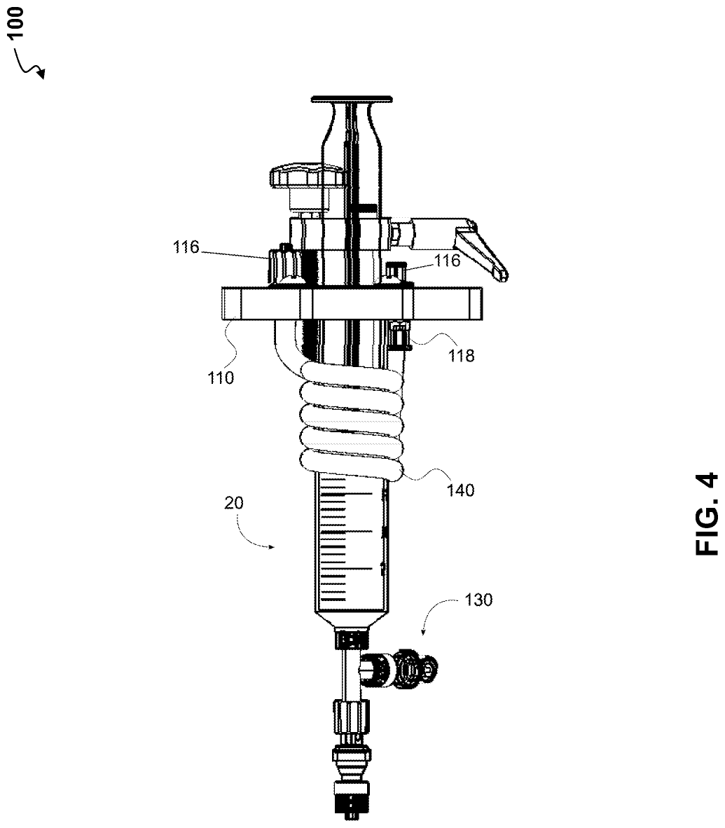

In reference to FIG. 4, each end of the hose 140 may be attached to a corresponding hose attachment member 116 on the base platform 110, allowing the present pressure adjustment system 100 to carry the hose 140 with the disposable syringe 20. The base platform 110 may have a valve assembly 130 attachment member 118 for receiving the valve assembly 130. The hose 140 is configured to be attached to the output of the valves, in some embodiments. In other embodiments, the hose 140 is configured to be attached directly to the syringe 20, such as in configurations where the valves are not used.

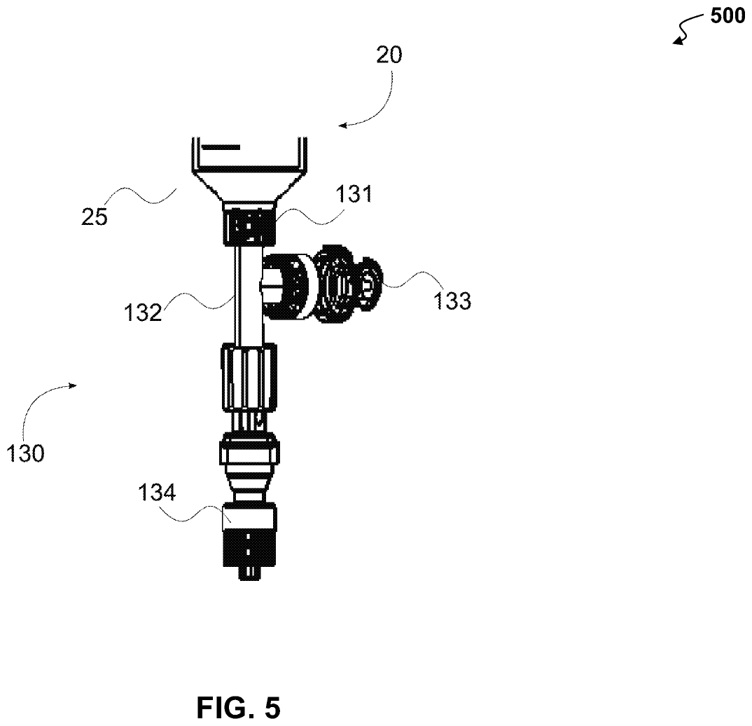

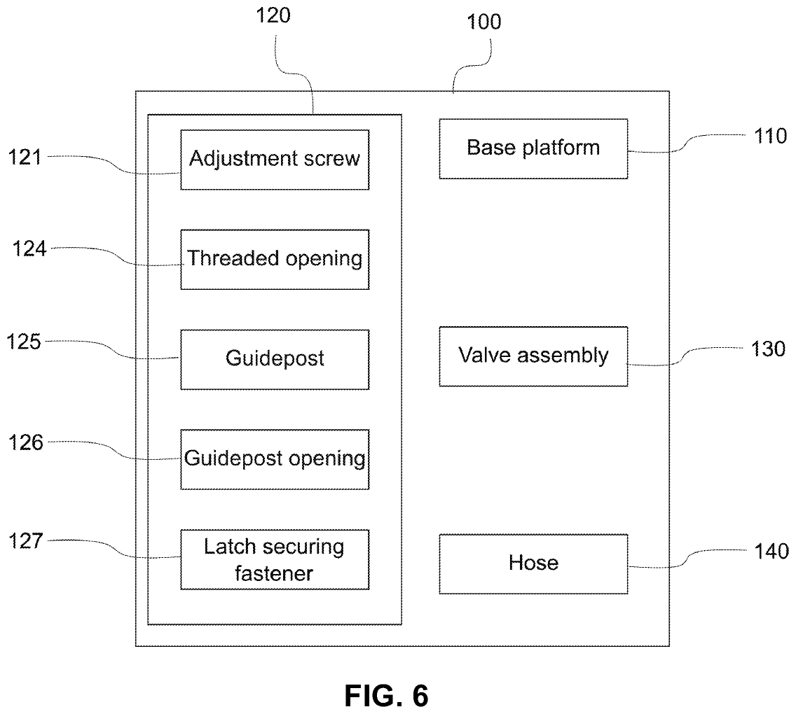

With respect to FIG. 5, the valve assembly 130 may be configured to be secured to a syringe barrel end 25 of the disposable syringe 20 using an adapter 131. The adapter 131 may be secured to the syringe barrel end 25 via a locking connector, such as a locking luer connector or any fittings that are securely joined by means of a tabbed hub on the female fitting which screws into threads in a sleeve on the male fitting. The locking connector may include a tapered plug and receptacle which are configured to form a friction fit when pressed together. The locking connector latches these parts together with an additional twist of the tapered plug and receptacle relative to each other. Other connectors for the adapter 131 and the syringe barrel end 25 are possible and contemplated. Once secured, air may travel into the syringe barrel of the syringe 20 through a first check valve 133 through a main shaft 132. Air may travel out of the valve assembly 130 through the main shaft 132 and out the second check valve 134. The first check valve 133 may be open to the atmosphere and the second check valve 134 may be connected to the device under test when the disclosed device is in use. The second check valve 134 may be connected to the device under test via the hose (ref. no. 140, FIG. 4) in some embodiments.

FIG. 6 is a high-level block diagram of the present pressure adjustment system 100. The present pressure adjustment system 100 may include a base platform 110, a latch assembly 120, a valve assembly 130, and a hose 140. The latch assembly 120 may include an adjustment screw 121, at least one threaded opening 124, a guidepost 125, a guidepost opening 126, and a latch securing fastener 127.

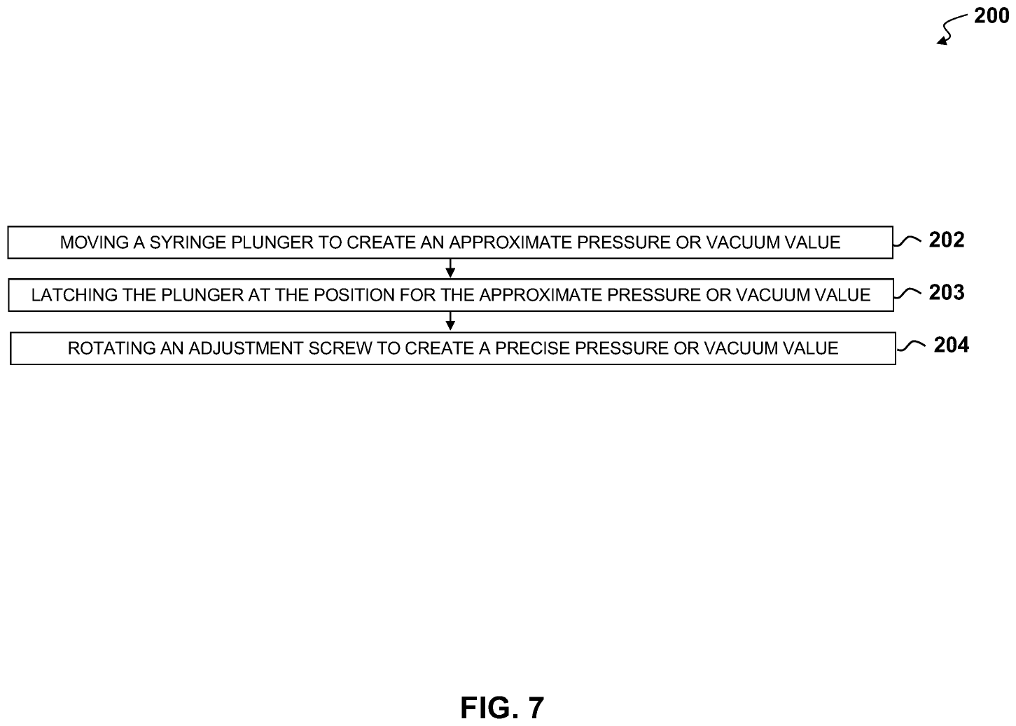

FIG. 7 is a flow chart of a method 200 for making fine adjustments to a syringe, for example, in small increments according to one embodiment. The method 200 may include moving a syringe plunger to create an approximate pressure or vacuum value (step 202). The method 200 may then include latching the syringe plunger in position for the approximate pressure or vacuum value (step 203). In one embodiment, the position may be an initial position based on an estimation by a user. The method 200 may then include rotating an adjustment screw to create a precise pressure or vacuum value (step 204). That is, once the user has adjusted the pressure or vacuum value to an approximate level, they may make slight or more precise adjustments to the pressure or vacuum value using the adjustment screw.

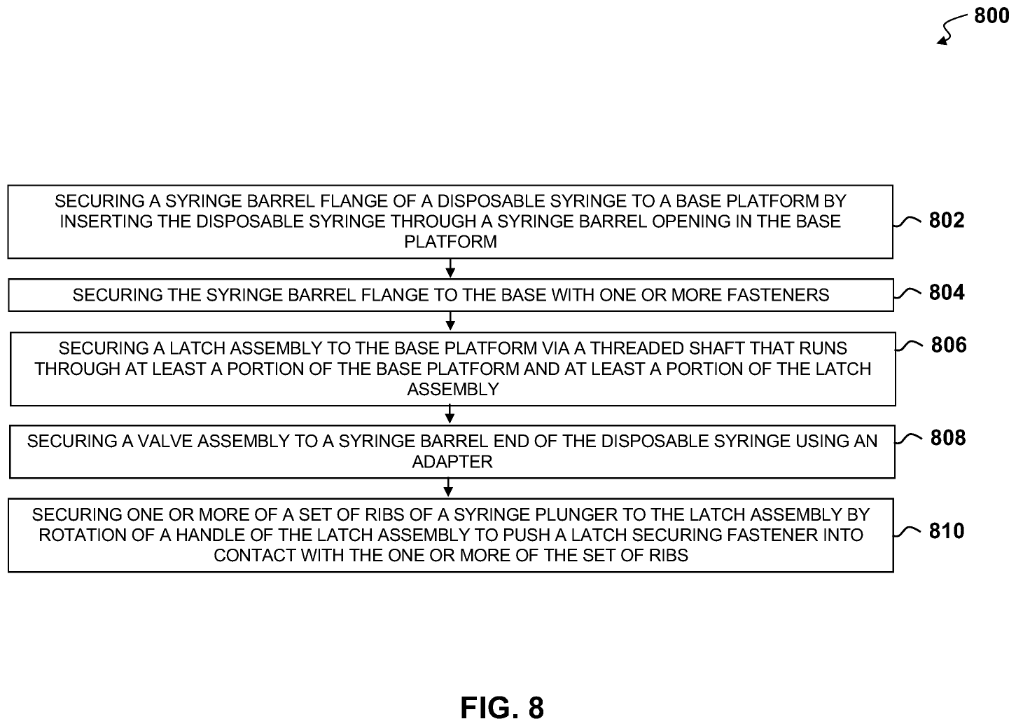

FIG. 8 is a flow chart for a method 800 for setting up the syringe holder system according to one embodiment. The method 800 may include securing a syringe barrel flange of a disposable syringe to a base platform by inserting the disposable syringe through a syringe barrel opening in the base platform (step 802). The method 800 may then include securing the syringe barrel flange to the base platform with one or more latch securing fasteners (step 804). The method 800 may then include securing a latch assembly to the base platform via a threaded shaft that runs through at least a portion of the base platform and at least a portion of the latch assembly (step 806). The method 800 may then optionally include securing a valve assembly to a syringe barrel end of the disposable syringe using an adapter (step 808).

For small test volume applications and embodiments, the valves may not be involved: that is, the syringe plunger would be depressed to an approximate pressure value, the lock would be engaged, and the knob would then be used to fine tune the pressure value. In larger volume applications, the syringe plunger would be moved back and forth to create a pumping action to inflate the test volume to a first approximate pressure, then the syringe plunger would be depressed to a closer approximate pressure and locked, then the knob would be utilized to adjust to the final pressure. The method 800 may then include securing one or more of a set of ribs of a syringe plunger to the latch assembly by rotation of a handle of the latch assembly to push a latch securing fastener into contact with the one or more of the set of ribs (step 810).

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention.

Further, it is intended that the scope of the present invention is herein disclosed by way of examples and should not be limited by the particular disclosed embodiments described above.

What is claimed is:

1. A system comprising:
a base platform defining a syringe flange opening configured to receive a syringe barrel flange of a syringe; and
a latch assembly configured to movably attach to the base platform via an adjustment screw threaded between the base platform and the latch assembly, wherein a distance between the base platform and the latch assembly is changed based on a rotation of the adjustment screw relative to the latch assembly, the latch assembly comprising:
a handle configured to rotate relative to the latch assembly; and
a latch securing fastener configured to engage a rib of a syringe plunger by rotation of the handle; and
wherein the rib of the syringe plunger is configured to be secured to the latch assembly via rotation of the handle of the latch assembly; and
wherein rotation of the adjustment screw is configured to generate a precise pressure within the syringe by moving the syringe plunger relative to the syringe barrel flange, when the rib of the syringe plunger is secured to the latch assembly.

2. The system of claim 1, wherein an increase in the distance between the base platform and the latch assembly is configured to decrease a pressure value of the syringe.

3. The system of claim 2, wherein a decrease in the distance between the base platform and the latch assembly is configured to increase a pressure value of the syringe.

4. The system of claim 1, further comprising:
a guidepost further configured to secure the base platform to the latch assembly, wherein the guidepost runs through a guidepost opening of the base platform and a guidepost opening of the latch assembly.

5. The system of claim 1, the rib of the syringe plunger is configured to be released via rotation of the handle of the latch assembly.

6. The system of claim 1, wherein the handle is configured to rotate relative to the latch assembly to move a latch securing fastener at least one of: toward the rib and away from the rib.

7. The system of claim 1, wherein the latch securing fastener comprises threads configured to mate with threads disposed in a portion of the latch assembly.

8. The system of claim 1, wherein the adjustment screw comprises a threaded shaft that runs through at least a portion of the base platform and at least a portion of the latch assembly.

9. The system of claim 1, further comprising:
a hose configured to be attached to an end of the syringe; and
a hose attachment member on the base platform, wherein the hose attachment member is configured to receive an end of the hose.

10. The system of claim 1, further comprising:
a valve assembly configured to be attached to an end of the syringe; and
a valve assembly attachment member on the base platform, wherein the valve assembly attachment member is configured to receive a portion of the valve assembly.

11. A method comprising:
securing a syringe barrel flange of a syringe to a base platform;
securing a latch assembly to the base platform via an adjustment screw threaded between at least a portion of the base platform and at least a portion of the latch assembly;
securing a rib of a syringe plunger of the disposable syringe to the latch assembly; and
rotating the adjustment screw to adjust a distance between the syringe plunger and the syringe barrel flange to generate a precise pressure within the syringe.

12. The method of claim 11, wherein rotation of the adjustment screw adjusts a distance between the base platform and the latch assembly.

13. The method of claim 11, wherein securing the rib of the syringe plunger of the disposable syringe to the latch assembly further comprises:
rotating a handle relative to the latch assembly, wherein a latch securing fastener is configured to engage the rib of the syringe plunger by rotation of the handle.

14. The method of claim 11, further comprising:
decreasing a pressure value of the syringe by increasing a distance between the base platform and the latch assembly.

15. The method of claim 11, further comprising:
increasing a pressure value of the syringe by decreasing a distance between the base platform and the latch assembly.

16. The method of claim 11, further comprising:
releasing the rib of the syringe plunger of the disposable syringe to the latch assembly.

17. The method of claim 11, further comprising:
detaching a valve assembly from a valve assembly attachment member on the base platform; and
securing the valve assembly to an end of the syringe.

18. The method of claim 11, further comprising:
detaching a hose from a hose attachment member on the base platform; and
securing the hose to an end of the syringe.

19. A method comprising:
securing a syringe barrel flange of a syringe to a base platform;
securing a latch assembly to the base platform via an adjustment screw threaded between at least a portion of the base platform and at least a portion of the latch assembly;
moving a syringe plunger relative to the syringe barrel flange to a distance between the syringe plunger and the syringe barrel flange to generate an approximate pressure within the syringe;
securing a rib of a syringe plunger of the disposable syringe to the latch assembly; and
rotating the adjustment screw to further adjust the distance between the syringe plunger and the syringe barrel flange to generate a precise pressure within the syringe.

20. The method of claim 19, wherein rotation of the adjustment screw adjusts a distance between the base platform and the latch assembly.
